(19) United States
(12) Patent Application Publication
(10) Pub. No.: US 2018/0317999 A1
(43) Pub. Date: Nov. 8, 2018

(54) TISSUE ABLATION SYSTEM

(71) Applicant: Intekplus Co., Ltd., Daejeon (KR)

(72) Inventors: Sung-Soo PARK, Seoul (KR); Young-Jae WON, Cheongju-si (KR); Seung-Rag LEE, Sejong-si (KR); Byung-Jun PARK, Sejong-si (KR); Byung-Yeon KIM, Cheongju-si (KR); Sang-Kyeong PARK, Sejong-si (KR); Hyeon-Jin BANG, Danyang-gun (KR)

(73) Assignee: Intekplus Co., Ltd., Daejeon (KR)

(21) Appl. No.: 15/772,958

(22) PCT Filed: Mar. 30, 2016
(86) PCT No.: PCT/KR2016/003243

§ 371 Date: May 2, 2018

(30) Foreign Application Priority Data
Nov. 3, 2015 (KR) 10-2015-0153505

(51) Int. Cl. A61B 18/14; A61B 5/00; A61B 17/32

(52) U.S. Cl. CPC A61B 18/1445; A61B 5/0079; A61B 2018/00982; A61B 5/0066; A61B 2017/320069; A61B 17/320092

(57) ABSTRACT

A tissue excising system includes: a tissue ablation device including an ablation unit ablate a body tissue, and an optical signal transmission module; an image generating unit including a light source configured to provide an optical signal to the optical signal transmission module and an optical interferometer configured to receive an optical signal reflected from the body tissue from the optical signal transmission module; and a display unit configured to display an image by receiving an optical image signal from the image generating unit. The optical signal transmission module line-scans the body tissue by penetrating the optical signal from the light source to the body tissue. The optical interferometer generates the optical image signal by applying optical coherence to the optical signal reflected from the body tissue by line-scanning, and the display unit images an inside of the body tissue, using the optical image signal.

### TECHNICAL FIELD

The present invention relates to a tissue excising system and, more particularly, to a tissue excising system that can minimize damage due to unexpected ablation or a blood vessel in an operation by allowing for visually observing whether there is a blood vessel and the sizes of blood vessels through an image showing the inside of a tissue to be ablated by line-scanning human body tissues even without a camera module that is inserted into a human body.

### BACKGROUND ART

An operation, which in medical terminology, means a surgery curing a disease by incising or ablating a skin, a mucous membrane, or other tissues with medical tools. An abdominal operation of medial operations is an operation that cuts and opens the abdominal cavity or the skin of person and then cures, receives a plastic surgery, or removes organs inside.

In an abdominal operation, a patient' s skin is incised to form a predetermined space through the skin and tissues and then operation is performed in the space, so there is a large scar remaining and recovery after the operation is slow, so recently, laparoscopy has attracted attention as an alternative to conventional surgery.

Laparoscopy, is a type of operation wherein a hole is formed at the position of a part of a patient to be operated on, a laparoscope is inserted through the hole, and then the operation is performed observing the part in the abdominal cavity. Laparoscopy is widely used in the fields of various internal and surgical operations, urology, and obstetrics and gynecology. Laparoscopy has many advantages such as a short recovery period, generation of a small scar, and reduction of pain and danger of infection, as compared with the existing abdominal operation, so development thereof has progressed rapidly after starting with cholecystectomy in 1990.

Laparoscopy has been applied to almost all surgical operations including colon cancer surgery, stomach cancer surgery, hernia surgery, liver resection, and thyroid surgery, which is 20˜40% of all operation and is expected to reach 80% of all operations in the future.

A laparoscope is a kind of equipment for visually diagnosing the internal organs of a body and is usually configured to make it possible to observe image information, which is detected by a small camera by inserting a device equipped with the small camera into a body, through an external monitor.

Since the positions and sizes of blood vessels existing in tissues to be ablated are very different, depending on patients, and the information about the positions and sizes cannot be known, laparoscopy should be performed by estimating the positions of blood vessels such as arteries on the basis of the anatomical knowledge and experience of doctors, so the possibility of unexpectedly ablating a blood vessel is very high when ablating a tissue.

When a blood vessel is ablated during laparoscopy, it takes much time and effort to stop bleeding, so the condition of a patient and a doctor may become worse, and if severe, the patient even may die due to a large loss of blood, so it may be a fatal problem.

There have been various researches to solve this problem, but they are half measures for reducing problems by stopping bleeding, using energy such as ultrasonic waves after a blood is ablated rather than studies for preventing unexpected ablation of a blood vessel when ablating a tissue during laparoscopy.

Briteseed in U.S.A. has filed a patent application, titled “SURGICAL TOOL WITH INTEGRATED SENSOR” (WO2013/134411 A1) in 2013, which provides a method of determining whether there is a blood vessel in a tissue on the basis of information about the intensity of light by radiating infrared light into the tissue from the upper end of a tissue ablation device and a measuring the intensity of light passing through the tissue by a light collection sensor at the lower end of the tissue ablation device.

However, since the positions and sizes of blood vessels passing through tissues are very different, depending on patients, it is difficult to obtain the exact information about whether there is blood vessel and the sizes of blood vessels in a tissue only from the intensity information of an optical signal passing through the tissue. Further, if the device malfunctions, considerable danger may be caused to a patient during an operation. The possibility of unexpectedly ablating a blood vessel is about 3% in actual operations and the possibility of causing fatal damage is about 18% in these cases, so several billion dollars are consumed as the costs for treatment due to unexpected ablation of a blood vessel.

Further, according to the related art, light is widely radiated to a tissue to be observed and the intensity of light passing through the tissue is collected by a light collection device such as a CCD, so the manufacturing costs of dispensable modules are high, which is not economical.

### DISCLOSURE

### Technical Problem

An object of the present invention is to provide a tissue excising system that can line-scan a human body tissue while moving forward and backward an optical signal transmission module connected to an external monitor even without a camera module that is inserted into a body.

Another object of the present invention is to provide a tissue excising system that can minimize damage due to unexpected ablation of a blood vessel during various surgeries such as laparoscopy, thoracoscopy, robotic surgery, or abdominal surgery by displaying whether there is a blood vessel or an ablation part and a non-ablation part in scan images from images showing the internal tissues of a part to be ablated using a tissue ablation device.

Another object of the present invention is to provide a tissue excising system that allows a doctor to ablate only a necessary part during an operation while immediately recognizing whether a human body tissue held by an ablation unit is a normal part or an abnormal part during various surgeries such as laparoscopy, thoracoscopy, or robotic surgery by line-scanning the human body tissue such as a blood vessel while moving an optical signal transmission module forward and backward and generating scan images of a normal part and abnormal part of the human body tissue through an image generating unit and an image display unit.

### Technical Solution

A tissue excising system according to an embodiment of the present invention includes: a tissue ablation device including an ablation unit having a structure that can ablate a human body tissue and an optical signal transmission module disposed to be movable forward and backward in a longitudinal direction of the ablation unit; an image generating unit including a light source providing an optical signal to the optical signal transmission module and an optical interferometer receiving an optical signal reflected from the human body tissue from the optical signal transmission module; and an image display unit displaying an image by receiving an optical image signal from the image generating unit, in which the optical signal transmission module line-scans the human body tissue by providing an optical signal reflected from the human body tissue to the optical interferometer by moving forward and backward in the longitudinal direction while penetrating an optical signal from the light source to the human body tissue, the optical interferometer generates the optical image signal by applying optical coherence to an optical signal provided by line-scanning by the optical signal transmission module, and the image display units images an inside of the human body tissue, using the optical image signal.

The image generating unit and the image display unit may image an optical signal provided by line-scanning by the optical signal transmission module, using optical coherence tomography (OCT).

In an embodiment of the present invention, the optical signal transmission module may include: an optical fiber disposed in the ablation unit to be movable forward and backward in the longitudinal direction and receiving an optical signal from the light source; an optical lens disposed at a front end of the optical fiber and diffusing light penetrated through the optical fiber; and a first optical mirror attached to the optical lens and reflecting an optical signal diffused through the optical lens to the human body tissue, and the first optical mirror may have a structure focused on the human body tissue such that an optical signal penetrated through the optical fiber is line-scanned by forward and backward movement of the optical fiber.

Alternatively, the optical signal transmission module may include: an optical fiber having a shape with a front end diagonally cut, disposed in the ablation unit to be movable forward and backward in the longitudinal direction, and receiving an optical signal from the light source; and an optical lens disposed between the front end of the optical fiber and the human body tissue and incidenting an optical signal penetrated through the optical fiber into the human body tissue, and the optical lens may have a structure focused on the human body tissue such that an optical signal penetrated through the optical fiber is line-scanned by forward and backward movement of the optical fiber.

Alternatively, the optical signal transmission module may include: an optical fiber having a convex lens structure at a front end, disposed in the ablation unit to be movable forward and backward in the longitudinal direction, and receiving an optical signal from the light source; and an optical mirror spaced from the front end of the optical fiber at a predetermined angle and reflecting an optical signal passing through the optical fiber into the human body tissue, and the optical mirror has a structure focused on the human body tissue such that an optical signal penetrated through the optical fiber is line-scanned by forward and backward movement of the optical fiber

Alternatively, the optical signal transmission module may include: an optical signal having an optical lens at a front end, disposed in the ablation unit to be movable forward and backward in the longitudinal direction, and receiving an optical signal from the light source; and a metal core tube disposed in the longitudinal direction of the optical fiber to cover the optical fiber and protecting the optical fiber.

The tissue ablation device may further include: an actuator operating a forceps-action of the ablation unit and controlling forward and backward movement of the optical signal transmission module; and an extension part connecting the ablation unit and the actuator to each other and having a flexibly movable pipe structure.

Here, the actuator may include: an actuator body connected to the extension part; and a forward/backward moving body disposed in the actuator body, connected to a rear end of the optical signal transmission mode, and operating such that a front end of the optical signal transmission unit is moved forward and backward in the first housing.

The actuator further may include: a guide pipe coupled to the extension part and guiding the optical signal transmission module; a rotary knob coupled to an outer side of the guide pipe and controlling a rotational angle of the ablation unit by rotating the guide pipe; and a guide plate fitted on the optical signal transmission module and coupled to a rear end of the guide pipe, and when the rotary knob is rotated, the optical signal transmission module may be moved by the guide plate by a rotational angle of the guide pipe.

In an embodiment of the present invention, the ablation unit may further include: a first housing accommodating the optical signal transmission module; and a second housing coupled to the first housing to form forceps, and the first housing and the second housing may hold the human body tissue through a forceps-action.

Here, a second optical signal transmission module may be disposed to be movable forward and backward at a position corresponding to the optical signal transmission module in the second housing, the second optical signal transmission module may line-scan the human body tissue by providing a second optical signal reflected from the human body tissue to the optical interferometer while moving in the longitudinal direction alternately with the optical signal transmission module and incidenting a second signal from the light source to the human body tissue, the optical interferometer may generate a second optical image signal by apply optical coherence to a second signal provided by line-scanning by the second optical signal transmission module, and the image display unit may image the inside of the human body tissue, using the second optical image signal.

In an embodiment of the present invention, the first housing may have a penetrating hole at a surface holding the human body tissue and penetrating an optical signal provided from the optical signal transmission module, and the second housing may have a second penetrating hole at a surface holding the human body tissue and penetrating the second optical signal provided from the second optical signal transmission module.

In an embodiment of the present invention, the penetrating hole and the second penetrating hole may be sealed with a permeable material that penetrates light.

The tissue ablation device may further include: an actuator operating a forceps-action of the ablation unit and controlling forward and backward movement of the optical signal transmission module and the second optical signal transmission module; and an extension part connecting the ablation unit and the actuator to each other and having a flexibly movable pipe structure.

Here, the actuator may include: an actuator body connected to the extension part and having an operation button; a forward/backward moving body disposed in the actuator, connected to a rear end of the optical signal transmission module, and operating such that a front end of the optical signal transmission module moves forward and backward in the first housing; and a second forward/backward moving body disposed in the actuator body, connected to a rear end of the second optical signal transmission module, and operating such that a front end of the second optical signal transmission module moves forward and backward in the second housing, and the forward/backward moving body and the second forward/backward moving body may be separately or simultaneously operated when the operation button is operated.

The actuator may further include: a guide pipe coupled to the extension part and guiding the optical signal transmission module and the second optical signal transmission module; a rotary knob coupled to an outer side of the guide pipe and controlling a rotational angle of the ablation unit by rotating the guide pipe; and a guide plate separately fitted on the optical signal transmission module and the second optical signal transmission module and coupled to a rear end of the guide pipe, and when the rotary knob is rotated, the optical signal transmission module and the second optical signal transmission module may be moved by the guide plate by a rotational angle of the guide pipe.

In a tissue excising system according to another embodiment of the present invention, optical signal transmission modules are disposed in pairs in the first housing, and second optical signal transmission modules are disposed in pairs in the second housing.

Here, the pair of optical signal transmission modules may be separately or simultaneously moved forward and backward in the first housing by operation of the actuator, and the pair of second optical signal transmission modules may be separately or simultaneously moved in the second housing by operation of the actuator.

The pair of second optical signal transmission modules may line-scan the human body tissue by collecting second optical signals reflected from the human body tissue while moving in the longitudinal direction alternately with the pair of optical signal transmission modules.

### Advantageous Effects

According to the present invention, provided is a tissue excising system that allows a doctor to ablate only a necessary part during an operation while immediately recognizing whether a human body tissue held by an ablation unit is a normal part or an abnormal part during various surgeries such as laparoscopy, thoracoscopy, or robotic surgery by line-scanning the human body tissue such as a blood vessel while moving an optical signal transmission module and a second optical signal transmission module forward and backward and generating scan images of a normal part and abnormal part of the human body tissue through an image generating unit and an image display unit.

According to the present invention, it is possible to obtain images of a human body tissue through optical signal transmission modules detachably connected to an external expensive camera module even without mounting a camera module on the part that is inserted into a human body and there is no need for a camera module that is installed on the part that is inserted into a human body to obtain images of a human body, so the manufacturing cost of the tissue ablation device can be reduced, which is economical.

Further, the tissue ablation device of the present invention has a structure detachably connected to an external device for imaging a human body tissue, so when the tissue ablation device is damaged, it can be easily replaced and can immediately keep taking images, whereby efficiency of laparoscopy can be increased.

According to the present invention, it is possible to directly observe whether there is a blood vessel and the sizes of blood vessels from images displaying the internal structure of a tissue to be ablated, so it is possible to minimize damage due to unexpected ablation of a blood vessel during a surgery.

That is, by using a tissue ablation device including a module for imaging the inside of a tissue to check whether there is a blood vessel in an ablated tissue in laparoscopy, it is possible to prevent a doctor from unexpectedly ablating a blood vessel by providing images displaying the internal structure of a tissue to be ablated on a monitor, so it is possible to safely ablate a tissue.

Further, according to the present invention, a replaceable tissue ablation device, and hardware and a processor for imaging a human body tissue are configured as a main device, so high price competitiveness can be achieved.

### DESCRIPTION OF DRAWINGS

FIG. 1 is a perspective view schematically showing a tissue ablation device according to a first embodiment of the present invention.

FIG. 2 is an exploded perspective view schematically showing the tissue ablation device according to a first embodiment of the present invention.

FIG. 3 is a cross-sectional view schematically showing a first housing taken along line A-A of FIG. 1.

FIG. 4 is a bottom view schematically showing the first housing according to the first embodiment of the present invention, FIG. 5 is a view showing a light path of an optical signal radiated to a human body tissue from an optical signal transmission module, and FIG. 6 is a cross-sectional view schematically showing the first housing with a penetrating hole sealed with a permeable material.

FIGS. 7 to 9 are views schematically showing various modifications of the optical signal transmission module according to the first embodiment of the present invention.

FIG. 10 is a view schematically showing the configuration of an actuator according to an embodiment of the present invention.

FIG. 11 is a view schematically showing the operation of an ablation unit when the actuator is operated.

FIG. 12 is a view schematically showing the operation of the ablation unit when the actuator stops operating.

FIG. 13 is a view schematically showing the configuration of a tissue excising system according to a first embodiment of the present invention.

FIG. 14 is a view schematically showing the configuration of a tissue excising system according to a second embodiment of the present invention.

FIG. 15 is a perspective view schematically showing a tissue excising system according to a third embodiment of the present invention.

FIG. 16 is a cross-sectional view schematically showing a first housing taken along line X-X in FIG. 15.

FIG. 17 is a bottom view schematically showing the first housing according to the third embodiment of the present invention.

FIG. 18 is a view showing a light path of an optical signal radiated to a human body tissue from a pair of optical signal transmission modules according to the third embodiment of the present invention.

FIG. 19 is a view schematically showing the configuration of a tissue excising system according to a third embodiment of the present invention.

### MODE FOR INVENTION

A tissue ablation device according to an exemplary embodiment and a tissue excising system using the tissue ablation device are described hereafter with reference to the accompanying drawings.

### First Embodiment

A tissue excising system equipped with a tissue ablation device according to the first embodiment is described hereafter.

Tissue Ablation Device

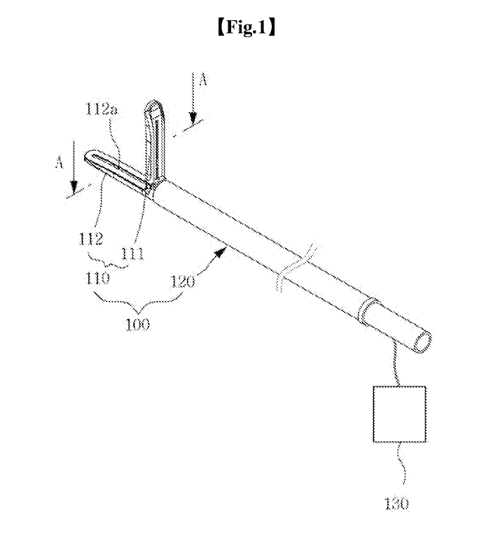

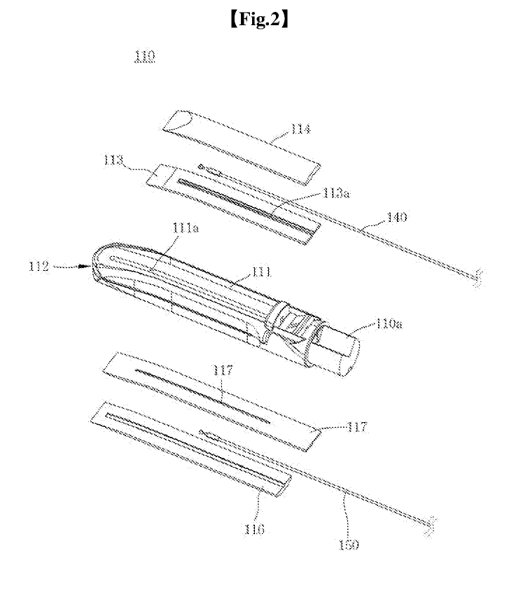

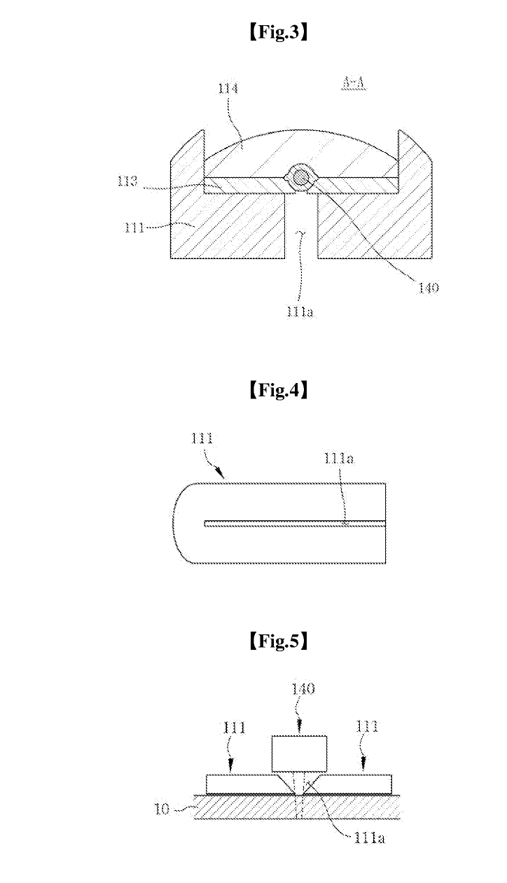

As shown in FIGS. 1 and 2, a tissue ablation device 100 includes an ablation unit 110, an extension part 120, and an optical signal transmission module 140.

The ablation unit 110 is inserted into a human body to hold or ablate a human body tissue 10. The optical signal transmission module 140 for line-scanning a part to be ablated from the human body tissue 10 is disposed in the ablation unit 110. A second optical signal transmission module 150 that is operated separately from the optical signal transmission module 140 may be further disposed in the ablation unit 110.

The optical signal transmission module 140 and the second optical signal transmission module 150 may be configured to display an ablation part and a non-ablation part of a human body tissue through an image display unit 230 by providing a reflected optical signal or a reflected second optical signal to an image generating unit 210 to be described below, by line-scanning both sides of the human body tissue held by the ablation unit 110 while alternately moving forward and backward in the longitudinal direction of the ablation unit 110.

The optical signal transmission module 140 and the second optical signal transmission module 150 are connected to the image generating unit 210, thereby separately providing optical signals reflected from a human body tissue to the image generating unit. The optical signal transmission module 140 and the second optical signal transmission module 150 are flexible to be able to provide light from the image generating unit to the human body tissue 10.

The optical signal transmission module 140 and the second optical signal transmission module 150 are disposed to be separately operated in the ablation unit 110.

The optical signal transmission module 140 is connected to an actuator 130 at the rear end of the extension part 120 to be controlled in forward/backward movement by the actuator 130. The actuator 130 is provided to automatically or manually control forward/backward movement of the optical signal transmission module 140. The actuator 130 will be described below.

The optical signal transmission module 140 has various structures. The optical signal transmission module 140 has the same structure as the second optical signal transmission module 150, so only the optical signal transmission module 140 is described to avoid repeated description.

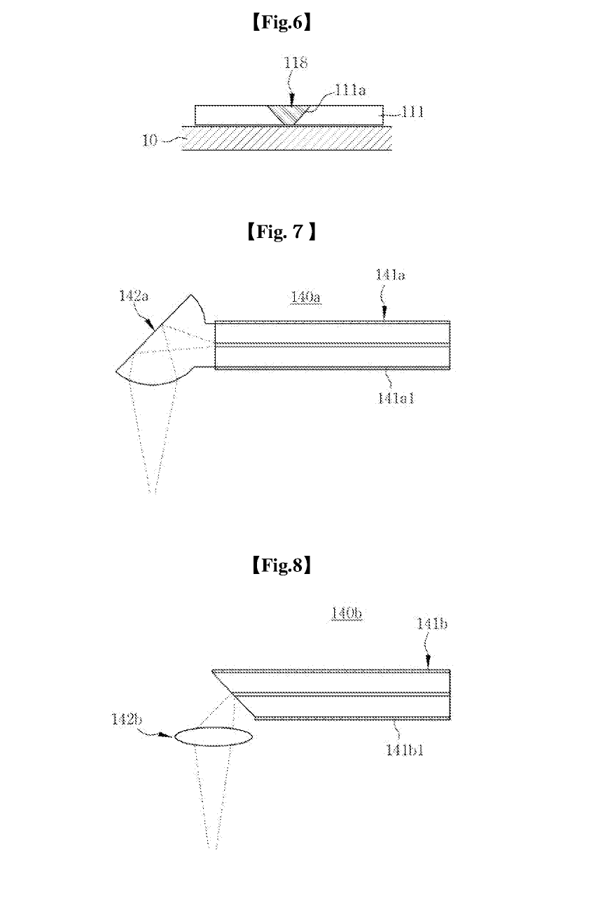

Various optical signal transmission modules 140 will be described with reference to FIGS. 7 to 9. In order to discriminate various types of optical signal transmission modules 140, they are discriminated by reference numerals 140a, 140b, and 140c in accordance with modifications.

An optical signal transmission module 140 according to a first modification is described with reference to FIG. 7. As shown in FIG. 7, the optical signal transmission module 140 is composed of an optical fiber 141a and an optical lens 142a.

The optical lens 142a is disposed at the front end of the optical fiber 141a. The rear end of the optical fiber 141a is connected to the actuator 130 at the rear end of the extension part 120. The optical fiber 141a is connected to the image generating unit 210. The image generating unit 210 provides an optical signal to the optical fiber 141a. The image generating unit 210 will be described below.

The optical fiber 141a is disposed to be movable forward/backward in a first guide groove 113a of a first housing 111 through the extension part 120 and provides an optical signal to the optical lens 142a. The optical lens 142a may be disposed in the first guide groove 113a to penetrate an optical signal to a penetrating hole 111a at a side of the first housing 111.

The optical fiber 141a is covered with a metal core tube 141a1 and protected by the metal core tube 141a1. The metal core tube 141a1 prevents the optical fiber 141a from bending and enables the optical fiber 141a to easily move forward/backward.

The optical lens 142a is formed by heating the front end of the optical fiber 141a into a lens and then grinding the front end at a predetermined angle. The optical lens 142a is provided to diffuse an optical signal that is light penetrated through the optical fiber. A first optical mirror 143a is disposed at the optical lens 142a. The first optical mirror 143a is provided to bend an optical signal penetrated through the optical fiber 141a at 90 degrees toward the human body tissue 10. That is, as shown in FIG. 7, the first optical mirror 143a may be attached to the optical lens 142a so that an optical signal penetrated through the optical fiber 141a incidents perpendicularly toward the human body tissue 10.

An optical signal transmission module 140b according to a second modification is described with reference to FIG. 8. As shown in FIG. 8, the optical signal transmission module 140b is composed of an optical fiber 141b and an optical lens 142b.

The optical fiber 141b is covered with a metal core tube 141a1 and protected by the metal core tube 141b1. The metal core tube 141b1 prevents the optical fiber 141b from bending and enables the optical fiber 141b to easily move forward/backward.

The front end of the optical fiber 141b is disposed in the first guide groove 113a of the first housing 111. The rear end of the optical fiber 141b is connected to the extension part 120. The front end of the optical fiber 141b is diagonally cut.

The optical lens 142b is disposed close to the front end of the optical fiber 141b. The optical lens 142b is provided to incident an optical signal penetrated from the optical fiber 141b perpendicularly toward the human body tissue 10.

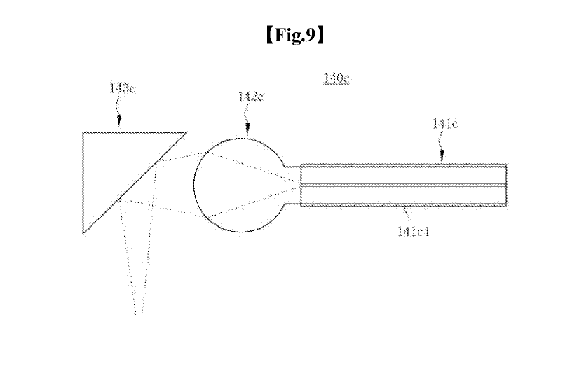

An optical signal transmission module 140c according to a third modification is described with reference to FIG. 9. As shown in FIG. 9, the optical signal transmission module 140c is composed of an optical fiber 141b and an optical mirror 143c.

The front end of the optical fiber 141c has the shape of a convex lens 142c. The optical fiber 141c, similar to the modifications described above, has a structure that can move in the longitudinal direction of the ablation unit 110.

The optical fiber 141c is covered with a metal core tube 141c1 and protected by the metal core tube 141c1. The metal core tube 141c1 prevents the optical fiber 141c from bending and enables the optical fiber 141c to easily move forward/backward.

The optical mirror 143c has a surface inclined at a predetermined angle, for example, 45°. The optical mirror 143c is disposed at a position where it reflects an optical signal from the convex lens 142c disposed at the front end of the optical fiber 141c so that the optical signal travels perpendicular to the human body tissue 10.

As described above, the second optical signal transmission module 150 has the same structure as the optical signal transmission module 140. The second optical signal transmission module 150 can perform the same function as the optical signal transmission module 140.

The second optical signal transmission module 150 can line-scan the human body tissue 10 by collecting second optical signals reflected from the human body tissue 10 while moving forward/backward in the longitudinal direction of a second housing 112 alternately with the optical signal transmission module 140.

The ablation unit 110 in which the optical signal transmission module 140 and the second optical signal transmission module 150 are disposed may have the following structure.

The ablation unit 110 includes an ablation body 110a, a first housing 111, a first guide member 113, a first housing cover 114, a second housing 112, a second guide member 117, and a second housing cover 116.

As shown in FIG. 1, the ablation body 110a is a member supporting the first housing 111 and the second housing 112 so that the housings can operate like forceps. The extension part 120 is connected to the rear end of the ablation body 110a.

The extension part 120 has a pipe shape that can flexibly move. The extension part 120 connects the ablation unit 110 that is inserted into a human body and the image generating unit 210 disposed at the outside to each other. The optical signal transmission module 140 and the second optical signal transmission module 150 may be disposed to be movable forward/backward in the extension part 120.

The optical signal transmission module 140 is built in the first housing 111. The front end of the first housing 111 may be rounded not to damage the human body tissue 10 when it is inserted into a human body.

The first guide member 113 that guides the optical signal transmission module 140 is disposed on the first housing 111 such that the optical signal transmission module 140 can move forward/backward. The first housing 111 has a penetrating hole 111a. The penetrating hole 111a is an opening formed through the surface of the first housing 111 for holding the human body tissue 10 to penetrate an optical signal from the optical signal transmission module 140. The penetrating hole 111a may be formed at the first housing 111 in the longitudinal direction of the first housing 111, that is, the front-rear direction of the optical signal transmission module 140.

As shown in FIG. 5, the penetrating hole 111a has a V-shaped cross-section. This is for making an optical signal from the optical signal transmission module 140 travel to the human body tissue 10 without diffusing.

The penetrating hole 111a is sealed with a permeable material that penetrates an optical signal from the optical signal transmission module 140. This is for preventing interference on forward/backward movement of the optical signal transmission module 140 by the human body tissue 10 or preventing damage to the optical signal transmission module 140 by a force that presses the optical signal transmission module 140, due to a portion of the human body tissue 10 that is put into the penetrating hole 111a by pressure that is applied to the human body tissue 10 when the ablation unit 110 holds the human body tissue 10, by filling the penetrating hole 111a with the permeable material.

The first guide member 113 is disposed on the side, which faces the second housing 112, of the first housing 111. A first guide groove 113a that guides the optical signal transmission module 140 is formed at the first guide member 113.

The first guide groove 113a is positioned to correspond to the penetrating hole 111a of the first housing 111. The first guide member 113 is provided to stably guide the optical signal transmission module 140 moving forward/backward.

The first housing cover 114 is disposed on the first housing 111 to cover the first guide member 113. The first housing cover 114 is provided to block light entering to the optical signal transmission module 140 from the outside and protect the optical signal transmission module 140.

The second housing 112 has the same structure as the first housing 111 and is connected to the ablation body 110a. The second housing 112 has a second penetrating hole 112a formed on the side facing the first housing 111 to penetrate an optical signal from the second optical signal transmission module 150.

The second penetrating hole 112a has a V-shaped cross-section, similar to the penetrating hole 111a described above. The second penetrating hole 112a is sealed with a permeable material that penetrates an optical signal from the optical signal transmission module 140.

The second guide member 117 and the second housing cover 116 are disposed on the second housing 112. The second guide member 117 is disposed on the side, which faces the first housing 111, of the second housing 112. A second guide groove 117a that guides the second optical signal transmission module 150 is formed on the second guide member 117.

The second guide member 117 may be disposed on the second housing 112 such that the second guide groove 117a is positioned to correspond to the second penetrating hole 112a. The second guide member 117 is provided to stably guide the second optical signal transmission module 150 moving forward/backward.

The second housing cover 116 is disposed on the second housing 112 to cover the second guide member 117. The second housing cover 116 is provided to block light entering to the second optical signal transmission module 140 from the outside and protect the second optical signal transmission module 150.

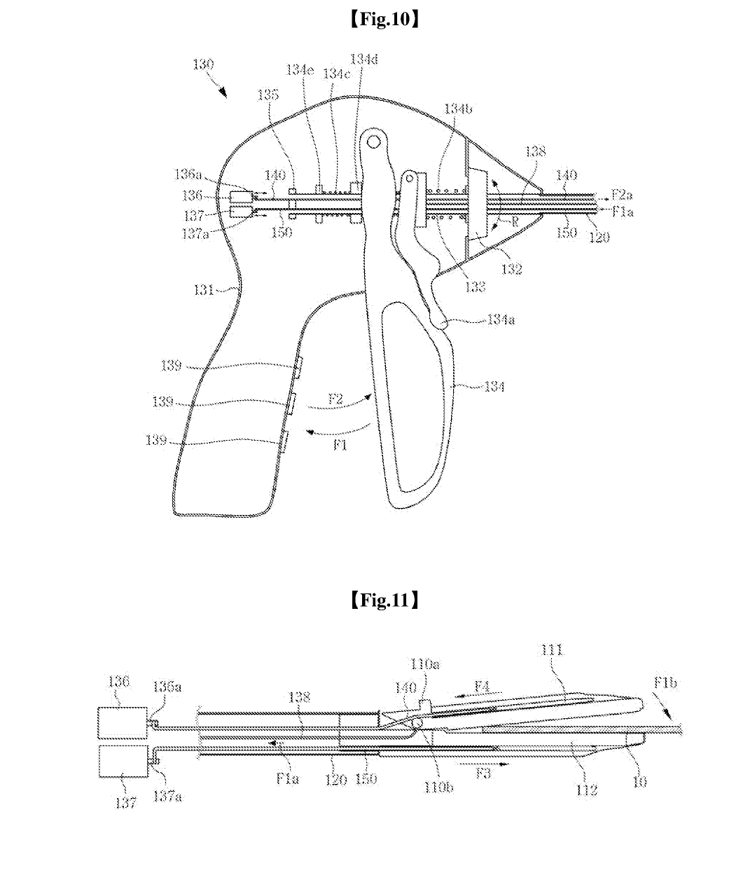

The actuator is described hereafter with reference to FIG. 10.

The actuator 130 is provided to automatically or manually control forward/backward movement of the optical signal transmission module 140. The actuator 130 is connected to the extension part to control the forceps-action of the ablation unit 10 and control the forward/backward movement of the optical signal transmission module 140 or the second optical signal transmission module 150.

As shown in FIG. 10, the actuator 130 includes an actuator body 131, a rotary knob 132, a guide pipe 133, a handle, a forward/backward moving body 136, and a second forward/backward moving body 137.

The rotary knob 132, the guide pipe 133, the forward/backward moving body 136, and the second forward/backward moving body 137 are disposed in the actuator body 131. A plurality of operation buttons 139 is disposed on the outer side of the actuator body 131. The operation buttons 139 are connected to the forward/backward moving body 136 and the second forward/backward moving body 137, thereby controlling the forward/backward movement directions and speeds of the optical signal transmission module 140 and the second optical signal transmission module 150. The number of the operation buttons 139 may be variously changed within the range that is apparent to those skilled in the art, depending on the way that the forward/backward moving body 136 and the second forward/backward moving body 137 operate the optical signal transmission module 140 and the second optical signal transmission module 150.

The guide pipe 133 is provided to guide the optical signal transmission module 140, the second optical signal transmission module 150, and an actuating wire 138 and is disposed in the actuator body 131. The extension part 120 is connected to the front end of the guide pipe 133. The guide pipe 133 has a pipe structure with open front and rear ends. The rotary knob 132, a handle body 134, a pulling member 134a, a first elastic member 134b, a second elastic member 134c, and a guide plate 135 are combined with the guide pipe 133.

The rotary knob 132 is combined with the guide pipe 133 and is positioned such that the outer side thereof is exposed to the outside of the actuator body 131. The rotary knob 132 is provided to control the rotational angle of the ablation unit 110 by rotating the guide pipe 133. When the rotary knob 132 is manually rotated by a predetermined angle, the guide pipe 133 combined with the rotary knob 132 is rotated by a predetermined angle and the ablation unit 110 connected to the guide pipe 133 through the extension part 120 is rotated by the rotational angle of the rotary knob 132 in the same direction as the guide pipe 133.

Even though the ablation unit 110 is rotated by rotation of the guide pipe 133, the optical signal transmission module 140 and the second optical signal transmission module 150 may not be rotated because they are disposed in the guide pipe 133. In order to prevent this problem, in the embodiment, the guide plate 135 is disposed at the rear end of the guide pipe 133 so that the optical signal transmission module 140 and the second optical signal transmission module 150 are rotated by the rotational angle of the guide pipe 133 when the rotary knob 132 is rotated.

The guide plate 135 has a plate structure through which the optical signal transmission module 140 and the second optical signal transmission module 150 separately pass, thereby guiding the optical signal transmission module 140 and the second optical signal transmission module 150 that move forward/backward. When the rotary knob 132 is rotated, the guide plate 135 limits the positions of the optical signal transmission module 140 and the second optical signal transmission module 150 by the rotational angle of the guide pipe 133 while being rotated by the guide pipe 133. When the rotary knob 132 is operated and the ablation unit 110 is rotated, the optical signal transmission module 140 and the second optical signal transmission module 150 can be rotated by the rotational angle of the ablation unit in the same direction as the ablation unit 110 by the guide plate 135.

The handle is provided to perform the forceps-action of the ablation unit 110. The handle is composed of the handle body 134, the pulling member 134a, the first elastic member 134b, the second elastic member 134c, the first elastic support 134d, and the second elastic support 134e.

The handle body 134 is hinged to the actuator body 131 to be movable along the guide pipe 133. The handle body 134 is rotated by a predetermined angle with respect to the actuator body 131.

The pulling member 134a is connected to the handle body 134. The pulling body 134a is hinged at an end to be rotatable by a predetermined angle with respect to the actuator body 131 and is movably coupled to the handle body 134 at the other end. The pulling member 134a is connected to the actuating wire 138 disposed in the guide pipe 133. The pulling body 134a pulls and releases the actuating wire 138 in cooperation with the handle body 134.

The handle body 134 and the pulling member 134a can pull the actuating wire 138 even with a small force due to the first elastic member 134b and the second elastic member 134c. When the force applied to the handle body 134 is removed, the first elastic member 134b and the second elastic member 134c allow the handle body 134 and the pulling member 134a to return to the initial positions.

In the embodiment, the first elastic member 134b has an end connected to the front end of the guide pipe 133 and the other end connected to the pulling member 134a. The second elastic member 134c has an end connected to the pulling member 134a and the other end connected to the rear end of the guide pipe 133. The second elastic member 134c is connected to the first elastic support 134d and the second elastic support 134e. The first elastic support 134d is coupled to the handle body 134 at the opposite side to the pulling body 134a. The first elastic support 134d has a structure that can move forward/backward along the guide pipe 133 in the movement direction of the handle body 134. The second elastic support 134e is fixed to the rear end of the guide pipe 133.

The operation structure of the ablation unit 110 according to the operation way of the handle is described hereafter with reference to FIGS. 10 to 12.

When a force is applied to the handle body 134 and the handle body 134 is rotated by a predetermined angle in the direction F1 with respect to the actuator body 131, the handle body 134 pulls the actuating wire 138 in the direction F1a in cooperation with the pulling member 134a.

When the handle body 134 is moved in the direction F1, the first elastic member 134b is moved and contracted in the direction F2a by the pulling member 134a and the second elastic member 134c is moved and extended in the direction F2a, so the actuating wire 138 can be easily pulled even by a small force due to the elasticity of the first elastic member 134b and the second elastic member 134c. As shown in FIG. 11, when the actuating wire 138 is pulled in the direction F1a by the handle body 134, a rotary shaft 110b of the ablation body connected with the actuating wire 138 is rotated by a predetermined angle, in which the first housing 111 is moved in the direction F1b and performs the forceps-action in cooperation with the second housing 112 t hold the human body tissue 10.

When the force applied to the handle body 134 is removed, the handle body 134 is moved in the direction F2 by the restoring force of the first elastic body 134b and the second elastic body 134c and returned to the initial position in cooperation with the pulling member 134a.

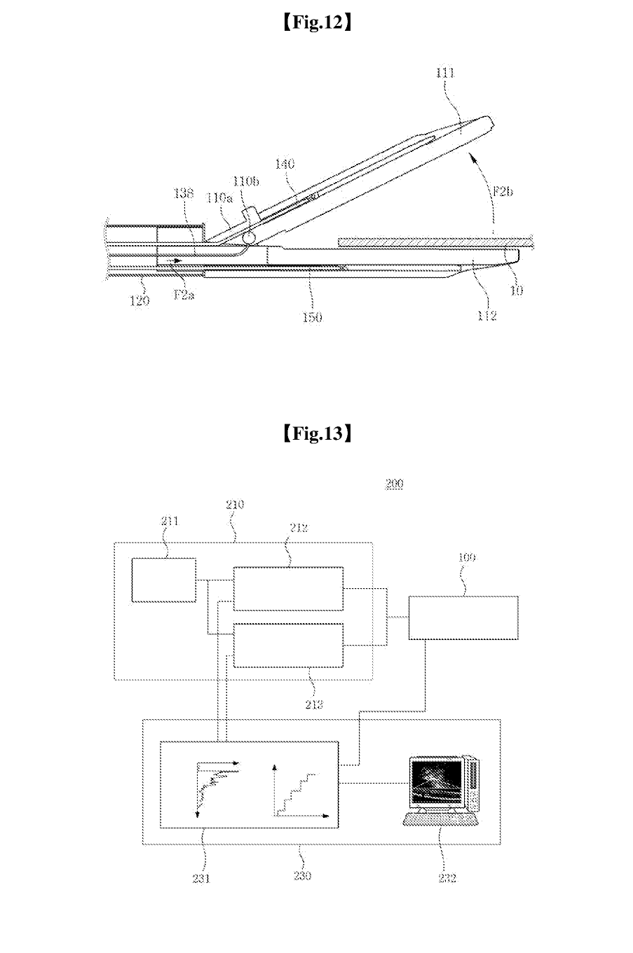

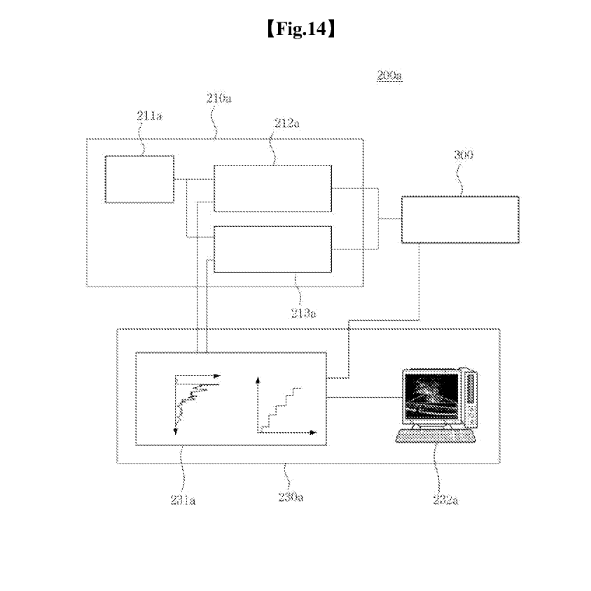

When the force applied to pull the actuating wire 138 by the pulling member 134a is removed, as shown in FIG. 12, the actuating wire 138 is moved in the direction F2a by the restoring force of an elastic spring fitted on the rotary shaft 110b of the ablation body. In this process, the first housing 111 is moved away from the second housing 112, so the held human body tissue 10 is released. A user can hold and release the human body tissue 10 by repeatedly operating the handle.

A user can scan the human body tissue 10 through the optical signal transmission module 140 and the second optical signal transmission module 150 by moving the optical signal transmission module 140 and the second optical signal transmission module 150 forward/backward through the forward/backward moving body 136 and the second forward/backward moving body 137 after holding the human body tissue 10 through the ablation unit 10 by operating the handle.

The forward/backward moving body 136 moves the optical signal transmission module 140 forward/backward and the second forward/backward moving body 137 moves the second optical signal transmission module 150 forward/backward. The forward/backward moving body 136 and the second forward/backward moving body 137 can be separately or simultaneously operated by operating the operation buttons 139.

In this embodiment, the forward/backward moving body 136 and the second forward/backward moving body 137 have the same structure. The forward/backward moving body 136 and the second forward/backward moving body 137 each include a driving motor and a forward/backward moving member. In the embodiment, the forward/backward moving member of the second forward/backward moving body 137 is referred to as a ‘second forward/backward moving member 137a’.

The forward/backward moving member is connected to the driving motor and moved forward/backward, depending on the rotational direction of the driving motor, by converting the rotational motion of the driving motor into a straight motion. For example, the forward/backward moving member is moved forward when a rotational shaft of the driving motor is rotated clockwise and is moved backward when the rotational shaft of the driving motor is rotated counterclockwise.

The optical signal transmission module 140 is connected to the forward/backward moving member 136a. When the forward/backward moving member 136a is moved forward/backward, the front end of the optical signal transmission module 140 is moved forward in the direction F3 or backward in the direction F4 in the first housing 111.

The second optical signal transmission module 150 is connected to the second forward/backward moving member 137a. When the second forward/backward moving member 137a is moved forward/backward, the front end of the second optical signal transmission module 150 is moved forward in the direction F3 or backward in the direction F4 in the second housing 112.

The forward/backward moving body 136 and the second forward/backward moving body 137 that have this structure can be automatically or manually operated. Since the forward/backward moving body 136 and the second forward/backward moving body 137 have the same operation structure, only the forward/backward moving body 136 is described hereafter to avoid repeated description.

According to the method of automatically operating the forward/backward moving body 136, when an operation button on the actuator 130 is pressed, the optical signal transmission module 140 is moved forward and/or backward along the first guide groove 113a and the optical signal transmission module 140 is operated to scan one time one line of the human body tissue 10 held by the ablation unit 110.

According to the method of automatically operating the forward/backward moving body 136, the optical signal transmission module provides an optical signal to the human body 10 while moving forward/backward at a constant speed, receives optical signals reflected from the human body tissue 10, and continuously provides the reflected optical signals to the image generating unit 210.

According to the method of manually operating the forward/backward moving body 136, a doctor may quickly move the optical signal transmission module 140 forward or backward by strongly pressing the operation button 139 of the actuator 130 manually or freely in accordance with his/her selection. Alternatively, a doctor may slowly move the optical signal transmission module 140 forward or backward by weakly pressing the operation button 139 of the actuator 130 and can control the movement distance of the optical signal transmission module 140 by changing the pressure. Accordingly, a doctor can control the scan distance of the human body tissue 10 as he/she wants.

Meanwhile, though not described in detail in the embodiment and not shown in the figures, a device such as an ultrasonic ablating tool or a high-frequency ablating tool is mounted at the joint between the first housing 111 and the second housing 112. Other than the ablating tool or the high-frequency ablating tool, various components that are used for ablating a tissue in laparoscopy can be mounted at the ablation unit 110 within a range that is apparent to those skilled in the art.

Tissue Excising System

A tissue excising system is described hereafter with reference to FIG. 13.

As shown in FIG. 13, a tissue excising system 200 according to an embodiment of the present invention includes a tissue ablation device 100, an image generating unit 210, and an image display unit 230. The tissue excising system 200 is a system that generates scan images to discriminate between an ablation part that is an abnormal tissue and a non-ablation part that is a normal tissue from each other in the human body tissue 10 held by the ablation unit 110 of the tissues ablation device 100 after the front end of the tissue ablation device 100 is inserted into a human body.

The tissue ablation device 100 applied to the tissue excising system 200 has the structure and function described above, so the tissue ablation device 100 is not described here.

The image generating unit 210 is detachably connected to the tissue ablation device 100. As shown in FIG. 13, the image generating unit 210 includes a light source 211, a first image generator 212, and a second image generator 213.

The light source 211 is a device that provides optical signals to the optical signal transmission module 140 and the second optical signal transmission module 150. The first image generator 212 and the second image generator 213 described in the embodiment perform the same function.

The first image generator 212 and the second image generator 213 may be optical interferometers to which optical coherence tomography (OCT) is applied.

The first image generator 212 is connected to the optical signal transmission module 140 outside a human body. The first image generator 212 generates a first optical image signal by receiving an optical signal reflected from the human body tissue 10 through the optical signal transmission module 140.

The first optical image signal is an optical coherence image generated by applying optical coherence to the optical signal that is reflected from a side of the human body tissue held by the ablation unit 110 and is provided through the optical signal transmission module 140.

The first optical image signal is provided to the image display unit 230. The optical signal means a signal that is penetrated to a human body tissue through the optical fiber of the optical signal transmission module 140 from the light source 211.

The second image generator 213 is connected to the second optical signal transmission module 150 outside a human body. The second image generator 213 generates a second optical image signal by receiving a second optical signal reflected from the human body tissue 10 through the second optical signal transmission module 150.

The second optical image signal is an optical coherence image generated by applying optical coherence to the optical signal that is reflected from another side of the human body tissue held by the ablation unit 110 and is provided through the second optical signal transmission module 150.

The second optical image signal is provided to the image display unit 230. The second optical signal means a signal that is penetrated to a human body tissue through the optical fiber of the second optical signal transmission module from the light source 211.

The image display unit 230 is connected to the image generating unit 210. The image display unit 230 of the optical signal transmission module 140 is provided to generate a scan signal of the human body tissue 10 by receiving an optical image signal according to a movement path and to image the human body tissue 10 into an ablation part and a non-ablation part from the scan signal. The ablation part is an abnormal tissue and the non-ablation part is a normal tissue.

The image display unit 230 includes a data processor 231 and a display device 232. The data processor 231 is connected to the image generating unit 210. The data processor 231 generates a first scan signal from the first optical image signal and generates a second scan signal from the second optical image signal.

The display device 232 is connected to the data processor 231. The display device 232 displays the human body tissue 10 into an ablation part and a non-ablation part from the first scan signal or second scan signal generated by the data processor 231. The first scan signal and the second scan signal are generated by the optical signal transmission module 140 and the second optical signal transmission module 150 alternately line-scanning the human body tissue 10 while at the position where the ablation unit 110 holds the human body tissue 10.

The optical signal transmission module 140 and the second optical signal transmission module 150 more accurately show an ablation part and a non-ablation part on the display device 232, for two sides of the human body tissue held by the ablation unit 110 on the basis of the first scan signal and the second scan signal by alternately line-scanning the human body tissue 10, whereby it is possible to induce correct determination by a doctor and prevent unexpected ablation of a blood vessel.

According to the present invention, scan signals are generated from continuous optical image signals collected in the line direction of the human body tissue 10 by the data processor 231, by receiving optical signals reflected from the human body tissue 10 through the optical signal transmission module 140 and the second optical signal transmission module 150 and by processing the optical signals into optical image signals through the image generating unit 210 disposed outside a human body even without a photographing sensor such as a camera on the tissue ablation device 100 that is inserted into the human body.

The scan signal means the slope of the graph that the x-axis is the reflection depth of the optical signal reflected from the human body tissue 10 and the y-axis is the backscattered intensity. A doctor can determine a part where the slope of the graph rapidly changes as an ablation tissue and a part where the graph change is not large as a non-ablation tissue and can ablate a blood vessel from the ablation tissue using the ablation unit 110 simultaneously with line-scanning by the optical signal transmission module 140, so convenience and accuracy of a surgery can be improved.

According to the present invention, it is possible to obtain images of a human body tissue through optical signal transmission modules detachably connected to an external expensive camera module even without mounting a camera module on the part that is inserted into a human body and there is no need for a camera module that is installed on the part that is inserted into a human body to obtain images of a human body, so the manufacturing cost of the tissue ablation device can be reduced, which is economical.

Further, the tissue ablation device of the present invention has a structure detachably connected to an external device for imaging a human body tissue, so when the tissue ablation device is damaged, it can be easily replaced and can immediately keep taking images, whereby efficiency of laparoscopy can be increased.

According to the present invention, it is possible to directly observe whether there is a blood vessel and the sizes of blood vessels from images displaying the internal structure of a tissue to be ablated, so it is possible to minimize damage due to unexpected ablation of a blood vessel during a surgery.

That is, by using a tissue ablation device including a module for imaging the inside of a tissue to check whether there is a blood vessel in an ablation tissue in laparoscopy, it is possible to prevent a doctor from unexpectedly ablating a blood vessel by providing images displaying the internal structure of a tissue to be ablated on a monitor, so it is possible to safely ablate a tissue.

Further, according to the present invention, a replaceable tissue ablation device, and hardware and a processor for imaging a human body tissue are configured as a main device, so high price competitiveness can be achieved.

### Second Embodiment

A tissue ablation device 100a according to a second embodiment of the present invention and a tissue excising system 200a using the tissue ablation device 100a are described hereafter.

The tissue ablation device 100a according to this embodiment is the same as the first embodiment described above in structure and function of the ablation unit 110, extension part 120, and actuator 130. Further, the installation positions and structure of an optical signal transmission module 140a and a second optical signal transmission module 150a are the same as those of the optical signal transmission module 140 and the second optical signal transmission module 150 of the first embodiment described above, so differences of the optical signal transmission module 140a and the second optical signal transmission module 150a from the first embodiment are described hereafter.

In the embodiment, the optical signal transmission module 140a is connected to a light source 211a of an image generating unit 210a and provides an optical signal from the light source 211a to a human body tissue. The tissue ablation device 100a according to the present invention can penetrate optical signals to a human body tissue while moving the optical signal transmission module 140a forward/backward in the longitudinal direction of an ablation unit, as in the first embodiment described above.

The second optical signal transmission module 150a is connected to a camera module 213a of the image generating unit 210a. The second optical signal transmission module 150a receive optical signal passing through a human body tissue from the optical signal transmission module 140a and provides the optical signals to the camera module 213a that is an external photographing device.

The tissue ablation device 100a according to the present invention can continuously receive optical signals passing through a human body tissues continuously penetrated from the optical signal transmission module 140a while moving the optical signal transmission module 150a in the movement direction of the optical signal transmission module 140a.

In the embodiment, the tissue excising system 200a can generate images of a human body tissue through an image generating unit 210a and an image display unit 230a, using optical signals collected by the operation of the tissue ablation device 100a.

The image generating unit 210a includes the light source 211a and the camera module 213a. The image generating unit 210a according to the embodiment can be applied to various optical systems such as an optical system using fluorescence, an optical system using general spectroscopy or Raman spectroscopy, and an optical system using a laser or an LED and these optical systems are well known in the art, so the optical technology of processing optical signals through the image generating unit 210 is not limited in detail in the embodiment.

The image display unit 230a includes a data processor 231a and a display device 232a. The data processor 231a is connected to the camera module 213a. The data processor 231a image-processes a human body tissue on the basis of optical image signals processed by the camera module 213a.

The display device 232a is connected to the data processor 231a. The display device 232a is provided to show images of the human body tissue 10 image-processed by the data processor 231a.

### Third Embodiment

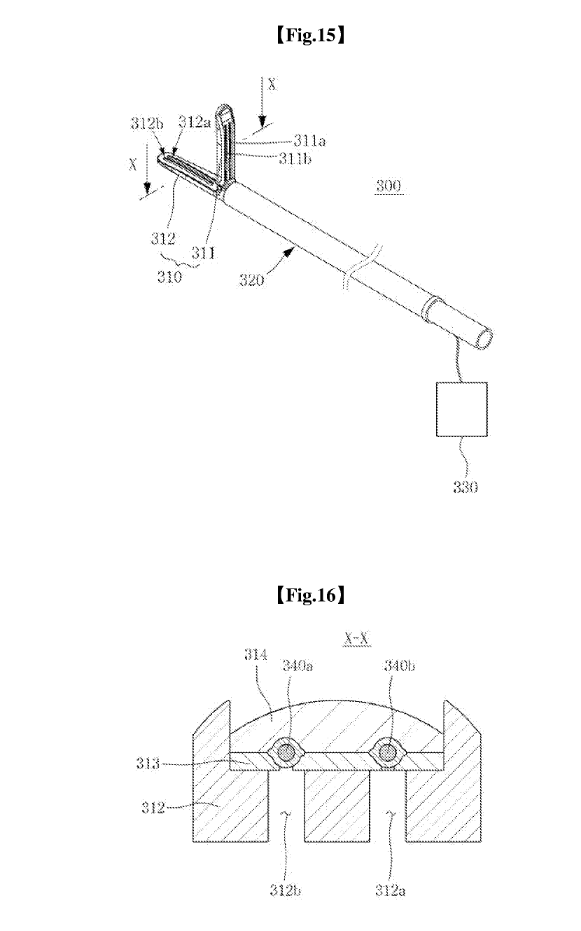

A tissue ablation device 300 according to a third embodiment of the present invention and a tissue excising system 200b using the tissue ablation device are described hereafter with reference to FIGS. 15 to 19.

As shown in FIGS. 15 to 18, the tissue ablation device 300 according to this embodiment include an ablation unit 310, an extension part 320, an actuator 330, a pair of optical signal transmission modules 340a and 340b, and a pair of second optical signal transmission modules.

The ablation unit 310, extension part 320, and actuator 330 of the embodiment have substantially the same functions as the ablation unit 110, extension part 120, and actuator 120 of the first embodiment described above. Further, the pair of optical signal transmission modules 340a and 340b and the pair of second optical signal transmission modules are the same in function and structure as the optical signal transmission module 140 and the second optical signal transmission module 150, but the arrangement structures are different.

The ablation unit 310, the pair of optical signal transmission modules 340a and 340b, and the pair of second optical signal transmission modules that are different in arrangement structure from the first embodiment described above are described hereafter.

The ablation unit 310 includes a first housing 311, a first guide member 313, a first housing cover 314, a second housing 312, a second guide member, and a second housing cover.

In the embodiment, the first housing 311, first guide member 313, first housing cover 314, second housing 312, second guide member, and second housing cover perform the same functions as the first housing 111, first guide member 113, first housing cover 114, second housing 112, second guide member 117, and second housing cover 116 of the first embodiment described above.

However, the structures of the first housing 311 and the second housing 312 on which the pair of optical signal transmission modules 340a and 340b and the pair of second optical signal transmission modules are respectively mounted are slightly different, so the structures of the first housing 311 and the second housing 312 are described hereafter.

A pair of penetrating holes 311a and 311b is formed at the first housing 311. The pair of penetrating holes 311a and 311b are arranged with a gap therebetween in the forward/backward movement direction of the pair of optical signal transmission modules 340a and 340b, on a surface for holding a human body tissue 10 of the first housing 311. Optical signals from the pair of optical signal transmission modules 340a and 340b penetrate the pair of penetrating holes 311a and 311b.

As shown in FIG. 18, the pair of penetrating holes 311a and 311b have a V-shaped cross-section. This is for making an optical signal from the pair of optical signal transmission modules 340a and 340b incident to the human body tissue 10 without diffusing.

The first guide member 313 is disposed on a side of the first housing 111. The side of the first housing 111 is the side facing the second housing 112. A pair of guide grooves (not shown) that communicate with the pair of penetrating holes 311a and 311b is formed at the first guide member 313. The optical signal transmission modules 340a and 340b are movably disposed on the first guide grooves (not shown), respectively. The first guide member 313 is provided to guide the pair of optical signal transmission modules 340a and 340b.

As shown in FIG. 16, the first housing cover 314 is disposed on the first housing 311 to cover the first guide member 313. The first housing cover 314 is provided to block light incideting into the pair of optical signal transmission modules 340a and 340b from the outside and to protect the pair of optical signal transmission modules 340a and 340b.

The second housing 312 has a pair of second penetrating holes 312a and 312b formed on the side facing the first housing 311 to penetrate optical signals from the pair of second optical signal transmission module. The second housing 312 has the same structure as the first housing 311, so the structure of the second housing 312 is not described in detail below to avoid repeated description.

As described above, the pair of optical signal transmission modules 340a and 340b and the pair of second optical signal transmission modules have the same structures and functions as the optical signal transmission module 140 and the second optical signal transmission module 150 of the first embodiment, but the numbers and positions are different, so the numbers and positions are described below.

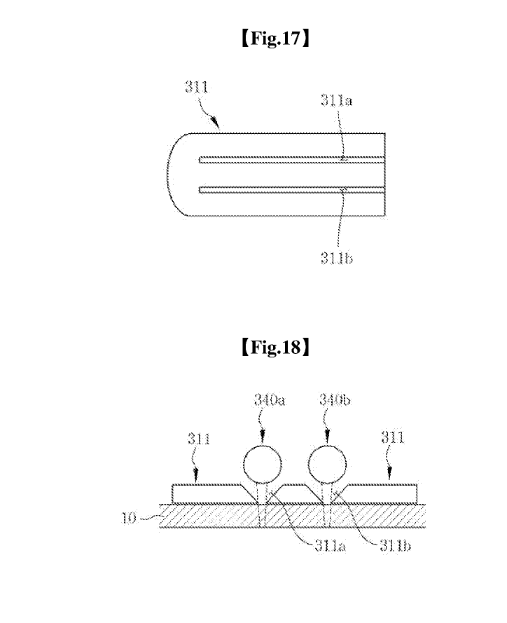

As shown in FIGS. 16 and 17, the pair of optical signal transmission modules 340a and 340b is disposed in the first housing 311 of the ablation unit 310 and spaced from each other in parallel with each other. The pair of optical signal transmission modules 340a and 340b may be arranged to provide optical signals to the human body tissue 10 through the pair of penetrating holes 311a and 311b formed at the first housing 311.

The pair of optical signal transmission modules 340a and 340b is provided to provide optical signals to a human body tissue and to line-scan the human body tissue by collecting optical signals reflected from the human body tissue while moving forward/backward in the longitudinal direction of the ablation unit 310.

The second optical signal transmission modules are arranged in parallel with each other in the second housing 312. The pair of second optical signal transmission modules may be arranged to provide second optical signals to the human body 10 through the penetrating holes 312a and 312b formed at the second housing 312. The pair of second optical signal transmission modules may be arranged to correspond to the pair of optical signal transmission modules 340a and 340b.

The pair of second optical signal transmission modules is provided to provide second optical signals to a human body tissue and to line-scan the human body tissue by collecting second optical signals reflected from the human body tissue while moving forward/backward in the longitudinal direction of the ablation unit 310.

The pair of optical signal transmission modules 340a and 340b and the pair of optical signal transmission modules provide reflected optical signals or reflected second optical signals to an image generating unit 210b to be described below, by line-scanning two sides of the human body tissue 10 held by the ablation unit 310 while alternately moving forward/backward in the longitudinal direction of the ablation unit 310. An image display unit 230b can image a human body tissue into an ablation part and a non-ablation part, using a first optical image signal and/or a second optical image signal converted by the image generating unit 210b.

That is, since the pair of optical signal transmission modules 340a and 340b and the pair of second optical signal transmission modules line-scan a human body tissue such as a blood vessel, and the image generating unit 210b and the image display unit 230b generate and show images, which a person can recognize, for an ablation part and a non-ablation part of the human body tissue such as blood vessel, a doctor can ablate only a necessary part in a surgery while immediately recognizing whether a human body tissue held by the ablation unit is a normal part or an abnormal part in various surgeries including laparoscopy, thoracoscopy, or a robotic surgery.

Meanwhile, though not described in detail in the embodiment and not shown in the figures, a device such as an ultrasonic ablation device or a high-frequency exicsor is mounted at the joint between the first housing 311 and the second housing 312. The ultrasonic ablating tool or high-frequency ablating tool may be disposed between the pair of optical signal transmission modules 340a and 340b on the first housing 311 and between the pair of second optical signal transmission modules on the second housing 312.

Other than the ablating tool or the high-frequency ablating tool, various components that are used for ablating a fine tissue such as a blood vessel in various surgeries such as laparoscopy, thoracoscopy, or a robotic surgery can be mounted at the ablation unit 310 within a range that is apparent to those skilled in the art.

Tissue Excising System

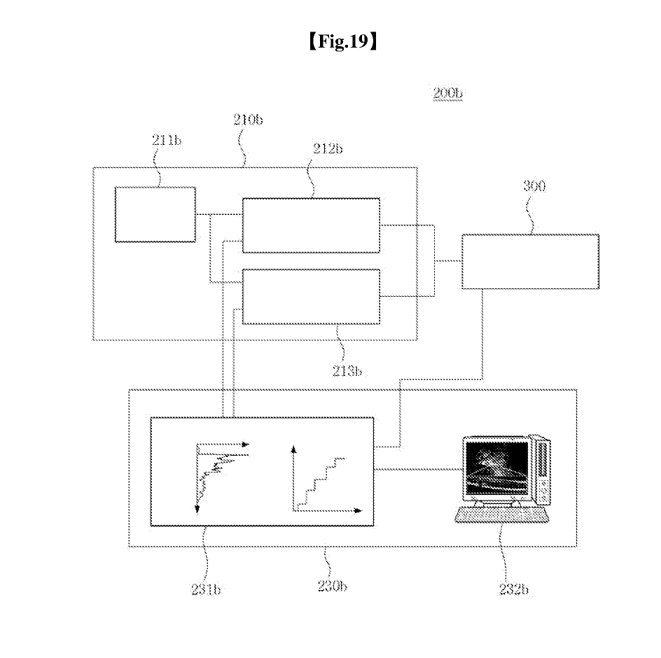

The tissue excising system according to the third embodiment of the present invention is described hereafter with reference to FIG. 19.

As shown in FIG. 19, a tissue excising system 200b according to an embodiment of the present invention includes a tissue ablation device 300, an image generating unit 210b, and an image display unit 230. The tissue excising system 200b is a system that generates images to discriminate between an ablation part that is an abnormal tissue and a non-ablation part that is a normal tissue from each other in the human body tissue 10 held by the ablation unit 310 of the tissues ablation device 300 after the front end of the tissue ablation device 300 is inserted into a human body.

The tissue excising system 200b according to the embodiment performs the same function as the tissue excising system 200 of the first embodiment described above. The tissue ablation device 300 applied to the tissue excising system 200b has the structure and function described above, so the tissue ablation device 300 is not described here.

As shown in FIG. 19, the image generating unit 210b includes a light source 211b, a first image generator 212b, and a second image generator 213b. The light source 211b is a device that provides optical signals to the pair of optical signal transmission modules 340a and 340b and the pair of second optical signal transmission modules.

The first image generator 212b and the second image generator 213 of the image generating unit 210b may be optical interferometers to which optical coherence tomography (OCT) is applied.

The first image generator 212b generates a first optical image signal by apply optical coherence to an optical signal reflected from the human body tissue 10 and provides the first optical image signal to the image display unit 230b. The second image generator 213b generates a second optical image signal by apply optical coherence to a second optical signal reflected from the human body tissue 10 and provides the second optical image signal to the image display unit 230b.

The first image generator 212b and second image generator 213b described in the embodiment perform the same functions as the first image generator 212 and the second image generator 213 of the first embodiment described above, so they are not described in detail.

The image display unit 230 is connected to the image generating unit 210b. The image display unit 230b generates scan signals of the human body tissue 10 by receiving the first optical image signal and the second optical image signal and images the human body tissue 10 into an ablation part and a non-ablation part on the basis of the scan signals. The ablation part is an abnormal tissue and the non-ablation part is a normal tissue.

The image display unit 230b includes a data processor 231b and a display device 232b. The data processor 231b is connected to the image generating unit 210b. The data processor 231b generates a first scan signal from the first optical image signal and generates a second scan signal from the second optical image signal.

The display device 232b is connected to the data processor 231b. The display device 232b images the human body tissue 10 into an ablation part and a non-ablation part from the first scan signal or second scan signal generated by the data processor 231b. The first scan signal and the second scan signal are generated by the pair of optical signal transmission modules 340a and 340b and the pair of second optical signal transmission modules alternately line-scan the human body tissue 10 at the position where the ablation unit 110 holds the human body tissue 10.

The pair of optical signal transmission modules 340a and 340b and the pair of second optical signal transmission modules more accurately show an ablation part and a non-ablation part on the display device 232b, for two sides of the human body tissue held by the ablation unit 310 on the basis of the first scan signal and the second scan signal by alternately line-scanning the human body tissue 10, whereby it is possible to induce correct determination by a doctor and prevent unexpected ablation of a blood vessel.

According to the present invention, scan signals are generated from continuous optical image signals collected in the line direction of the human body tissue 10 by the data processor 231b, by receiving optical signals reflected from the human body tissue 10 through the pair of optical signal transmission modules 340a and 340b and the pair of second optical signal transmission modules and by processing the optical signals into optical image signals through the image generating unit 210b disposed outside a human body even without a photographing sensor such as a camera on the tissue ablation device 300 that is inserted into the human body.

The scan signal means the slope of the graph that the x-axis is the reflection depth of the optical signal reflected from the human body tissue 10 and the y-axis is the backscattered intensity. A doctor can determine a part where the slope of the graph rapidly changes as an ablation tissue and a part where the graph change is not large as a non-ablation tissue and can ablate a blood vessel from the ablation tissue using the ablation unit 310 simultaneously with line-scanning by the pair of optical signal transmission modules 340a and 340b, so convenience and accuracy of a surgery can be improved.

According to the present invention, it is possible to obtain images of a human body tissue through optical signal transmission modules detachably connected to an external expensive camera module even without mounting a camera module on the part that is inserted into a human body and there is no need for a camera module that is installed on the part that is inserted into a human body to obtain images of a human body, so the manufacturing cost of the tissue ablation device can be reduced, which is economical.

According to the present invention, a blood vessel to be ablated is line-scanned by the tissue ablation device 300 and an ablation part and a non-ablation part of the blood vessel are discriminated by scan images generated by line-scanning, so it is possible to minimize damage due to unexpected ablation of a blood vessel in a surgery.

Some embodiments of the present invention were shown and described, but it would be understood by those skilled in the art that the embodiments may be modified without departing from the spirit or scope of the present invention. The range of the present invention will be determined by claims and equivalents.

1. A tissue excising system comprising:
a tissue ablation device including an ablation unit having a structure that can ablate a human body tissue and an optical signal transmission module disposed to be movable forward and backward in a longitudinal direction of the ablation unit;
an image generating unit including a light source providing an optical signal to the optical signal transmission module and an optical interferometer receiving an optical signal reflected from the human body tissue from the optical signal transmission module; and
an image display unit displaying an image by receiving an optical image signal from the image generating unit,
wherein the optical signal transmission module line-scans the human body tissue by providing an optical signal reflected from the human body tissue to the optical interferometer by moving forward and backward in the longitudinal direction while penetrating an optical signal from the light source to the human body tissue,
the optical interferometer generates the optical image signal by applying optical coherence to an optical signal provided by line-scanning by the optical signal transmission module, and
the image display units images an inside of the human body tissue, using the optical image signal.

2. The tissue excising system of claim 1, wherein the image generating unit and the image display unit images an optical signal provided by line-scanning by the optical signal transmission module, using optical coherence tomography (OCT).

3. The tissue excising system of claim 1, wherein the optical signal transmission module includes:
an optical fiber disposed in the ablation unit to be movable forward and backward in the longitudinal direction and receiving an optical signal from the light source;
an optical lens disposed at a front end of the optical fiber and diffusing light penetrated through the optical fiber; and
a first optical mirror attached to the optical lens and reflecting an optical signal diffused through the optical lens to the human body tissue, and
the first optical mirror has a structure focused on the human body tissue such that an optical signal penetrated through the optical fiber is line-scanned by forward and backward movement of the optical fiber.

4. The tissue excising system of claim 1, wherein the optical signal transmission module includes:
an optical fiber having a shape with a front end diagonally cut, disposed in the ablation unit to be movable forward and backward in the longitudinal direction, and receiving an optical signal from the light source; and
an optical lens disposed between the front end of the optical fiber and the human body tissue and incidenting an optical signal penetrated through the optical fiber into the human body tissue, and
the optical lens has a structure focused on the human body tissue such that an optical signal penetrated through the optical fiber is line-scanned by forward and backward movement of the optical fiber.

5. The tissue excising system of claim 1, wherein the optical signal transmission module includes:
an optical fiber having a convex lens structure at a front end, disposed in the ablation unit to be movable forward and backward in the longitudinal direction, and receiving an optical signal from the light source; and
an optical mirror spaced from the front end of the optical fiber at a predetermined angle and reflecting an optical signal passing through the optical fiber into the human body tissue, and
the optical mirror has a structure focused on the human body tissue such that an optical signal penetrated through the optical fiber is line-scanned by forward and backward movement of the optical fiber.

6. The tissue excising system of claim 1, wherein the optical signal transmission module includes:
an optical fiber having an optical lens at a front end, disposed in the ablation unit to be movable forward and backward in the longitudinal direction, and receiving an optical signal from the light source; and
a metal core tube disposed in the longitudinal direction of the optical fiber to cover the optical fiber and protecting the optical fiber.

7. The tissue excising system of claim 1, wherein the tissue ablation device further includes:
an actuator operating a forceps-action of the ablation unit and controlling forward and backward movement of the optical signal transmission module; and
an extension part connecting the ablation unit and the actuator to each other and having a flexibly movable pipe structure.

8. The tissue excising system of claim 7,
wherein the actuator includes:
an actuator body connected to the extension part; and
a forward/backward moving body disposed in the actuator body, connected to a rear end of the optical signal transmission mode, and operating such that a front end of the optical signal transmission unit is moved forward and backward in the first housing.

9. The tissue excising system of claim 7, wherein the actuator further includes:
a guide pipe coupled to the extension part and guiding the optical signal transmission module;
a rotary knob coupled to an outer side of the guide pipe and controlling a rotational angle of the ablation unit by rotating the guide pipe; and
a guide plate fitted on the optical signal transmission module and coupled to a rear end of the guide pipe, and
when the rotary knob is rotated, the optical signal transmission module is moved by the guide plate by a rotational angle of the guide pipe.

10. The tissue excising system of claim 1, wherein the ablation unit further includes:
a first housing accommodating the optical signal transmission module; and
a second housing coupled to the first housing to form forceps,
wherein the first housing and the second housing holds the human body tissue through a forceps-action.

11. The tissue excising system of claim 10, wherein a second optical signal transmission module is disposed to be movable forward and backward at a position corresponding to the optical signal transmission module in the second housing,
the second optical signal transmission module line-scans the human body tissue by providing a second optical signal reflected from the human body tissue to the optical interferometer while moving in the longitudinal direction alternately with the optical signal transmission module and incidenting a second signal from the light source to the human body tissue,
the optical interferometer generates a second optical image signal by apply optical coherence to a second signal provided by line-scanning by the second optical signal transmission module, and
the image display unit displays the inside of the human body tissue, using the second optical image signal.

12. The tissue excising system of claim 11, wherein the first housing has a penetrating hole at a surface holding the human body tissue and penetrating an optical signal provided from the optical signal transmission module, and
the second housing has a second penetrating hole at a surface holding the human body tissue and penetrating the second optical signal provided from the second optical signal transmission module.

13. The tissue excising system of claim 12, wherein the penetrating hole and the second penetrating hole are sealed with a permeable material that penetrates light.

14. The tissue excising system of claim 11, further comprising:
an actuator operating a forceps-action of the ablation unit and controlling forward and backward movement of the optical signal transmission module and the second optical signal transmission module; and
an extension part connecting the ablation unit and the actuator to each other and having a flexibly movable pipe structure.

15. The tissue excising system of claim 14, wherein the actuator includes:
an actuator body connected to the extension part and having an operation button;
a forward/backward moving body disposed in the actuator, connected to a rear end of the optical signal transmission module, and operating such that a front end of the optical signal transmission module moves forward and backward in the first housing; and
a second forward/backward moving body disposed in the actuator body, connected to a rear end of the second optical signal transmission module, and operating such that a front end of the second optical signal transmission module moves forward and backward in the second housing,
and the forward/backward moving body and the second forward/backward moving body are separately or simultaneously operated when the operation button is operated.

16. The tissue excising system of claim 15, wherein the actuator further includes:
a guide pipe coupled to the extension part and guiding the optical signal transmission module and the second optical signal transmission module;
a rotary knob coupled to an outer side of the guide pipe and controlling a rotational angle of the ablation unit by rotating the guide pipe; and
a guide plate separately fitted on the optical signal transmission module and the second optical signal transmission module and coupled to a rear end of the guide pipe, and
when the rotary knob is rotated, the optical signal transmission module and the second optical signal transmission module are moved by the guide plate by a rotational angle of the guide pipe.

17. The tissue excising system of claim 11, wherein the optical signal transmission module is disposed in pairs in the first housing, and
the second optical signal transmission module is disposed in pairs in the second housing.

18. The tissue excising system of claim 17, wherein the pair of optical signal transmission modules is separately or simultaneously moved forward and backward in the first housing by operation of the actuator, and
the pair of second optical signal transmission modules is separately or simultaneously moved in the second housing by operation of the actuator.

19. The tissue excising system of claim 18, wherein the pair of second optical signal transmission modules line-scans the human body tissue by collecting second optical signals reflected from the human body tissue while moving in the longitudinal direction alternately with the pair of optical signal transmission modules.
